(19) United States
(12) Patent Application Publication
(10) Pub. No.: US 2004/0133453 A1
(43) Pub. Date: Jul. 8, 2004

(54) Method and system for providing at home health care service

(76) Inventors: Jean-Philippe Jomini, Boca Raton, FL (US); Leigh A. Hough, Boca Raton, FL (US); Russell Zimmerman, Boca Raton, FL (US)

Correspondence Address: SACCO & ASSOCIATES, PA, P.O. BOX 30999, PALM BEACH GARDENS, FL, 33420-0999, US

(21) Appl. No.: 10/723,444

(22) Filed: Nov. 26, 2003

Related U.S. Application Data
(60) Provisional application No. 60/429,748, filed on Nov. 27, 2002.

(51) Int. Cl. G06F 17/60; A61B 5/00

(52) U.S. Cl. 705/2; 600/300

(57) ABSTRACT

A method for in-home monitoring which monitors at least one behavioral parameter associated with a person. The behavioral parameter can be compared to at least one pre-determined rule which is based upon a behavioral profile. The data representing the behavioral parameter can be forwarded from a sensing device (102) to a device interface (104) of a server (101) within the person's home. In one arrangement, the data can be wirelessly propagated to the device interface. An exception can be triggered if the behavioral parameter does not match the behavioral profile. The exception can be forwarded to a monitoring server (180) located outside the home and at least one action responsive to the exception can be initiated.

### CROSS REFERENCE TO RELATED APPLICATIONS

This Application is related to, and claims priority of U.S. provisional patent application No. 60/429,748, filed Nov. 27, 2002.

### BACKGROUND OF THE INVENTION

1. Technical Field

This invention relates to the field of medical care systems and, more particularly, to home care monitoring systems.

2. Description of the Related Art

As most people age, suffer from chronic diseases, or become handicapped physically or mentally, they generally prefer to live at home for as long as possible rather than move into a long term care institution or hospital. One option to extend a person's time at home before moving into a long term care institution is home care. As a general rule, with the proper care, most people can live out their lives at home without ever entering a care facility. In-home care services can be very expensive, however, especially in cases where continuous supervision is required.

In the United States, Medicare will cover a limited amount of home care expenses for some patients having certain non-permanent conditions, but Medicare will not cover long term home care expenses. When Medicare benefits have expired and there is little or no money available for a person's care, a decision is often made to use Medicaid. Medicaid will pay for certain aspects of home care, but only while it is more cost effective and more efficient than a nursing home, which is rarely the case. Thus, only those families with substantial assets or income are able to continue home care with professional services. Consequently, a lack of financial resources typically is the primary reason why most persons are unable to take advantage of in-home care services. Accordingly, a solution is needed to reduce the cost of home care and extend the amount of time a person who is elderly or ill can live at home.

### SUMMARY

The present invention relates to a method for in-home monitoring which monitors at least one behavioral parameter associated with a person. The behavioral parameter can be compared to at least one pre-determined rule which is based upon a behavioral profile. The data representing the behavioral parameter can be forwarded from a sensing device to a device interface of a server within the person's home. In one arrangement, the data can be wirelessly propagated to the device interface. An exception can be triggered if the behavioral parameter does not match the behavioral profile. The server can forward the exception to a monitoring system located outside the person's home and at least one action responsive to the exception can be initiated.

The behavioral profile can be empirically determined based upon behavioral patterns of the person. Moreover, the behavioral parameter can be analyzed using artificial intelligence implemented with an inference engine. The behavioral parameter can be an acoustic signal, a movement of a person, a location of a person, an opening or closing of a window, an opening or closing of a door, an activation or deactivation of a light, and/or an activation or deactivation of an appliance.

Further, environment parameters can be monitored. For example, an environment parameter can be compared to at least one pre-determined environment rule. Again, an exception can be triggered if the environment parameter correlates to an environment condition pre-defined to trigger the exception. The environment parameter can be a carbon monoxide level, a smoke level, a temperature, an amount of water intrusion, a moisture level, a power failure, a weather condition, an earthquake, an acoustic signal, an opening or closing of a window, an opening or closing of a door and/or or a detected motion.

A medical parameter can also be monitored. The medical parameter can be compared to at least one pre-determined medical rule. An exception can be triggered if the medical parameter correlates to a medical condition pre-defined to trigger the exception. The medical parameter can be a blood pressure, a pulse, a blood glucose level, a blood oxygen level, a weight, a heart rhythm, a brain wave, and/or a breathing pattern. A client-phone localized emergency call can be initiated, for example during a medical emergency. In one arrangement, a medication reminder can be generated, for instance to remind a client to take prescribed medication.

The invention also concerns a system for in-home monitoring. The system can include a sensor for monitoring a behavioral parameter associated with a person and generating correlating data. The sensor also can monitor environment parameters. For example, the sensor can be a microphone, a video camera, an infrared motion detector, a carbon monoxide detector, a smoke detector, a fire detector, a water intrusion detector, a power failure detector, a door contact or a window contact. The sensor also can monitor a physical attribute of a person, for example a blood pressure, a pulse, a blood glucose level, a blood oxygen level, a weight, a heart rhythm, a brain wave, and/or a breathing pattern. The system further can include roving robots which can monitor the behavioral parameters, the environment parameters or the physical attribute of the person.

The system also can include at least one processing device. A device interface can be provided for receiving the data and forwarding the data to the processing device. For example, the data can be wirelessly propagated to the device interface. A software application can be used to compare the data to a pre-determined rule which is based upon a behavioral profile. The software application can trigger an exception if the data correlates to a condition pre-defined to trigger the exception.

The system also can include a communication link for communicating with a monitoring station. The processing device can forward the exception to the monitoring station. The system can receive remote commands from the monitoring station. The remote commands can be transmitted over the communication link. The remote commands can control, for example, an appliance, a lamp, a sensor and/or a medical device. Further, client-phone localized emergency calls can be generated via a remote command, such as when a medical emergency is detected.

### BRIEF DESCRIPTION OF THE DRAWINGS

There are shown in the drawings, embodiments which are presently preferred, it being understood, however, that the invention is not limited to the precise arrangements and instrumentalities shown.

FIGS. 1A and 1B, taken together, represent a block diagram of a home care monitoring system that is useful for understanding the present invention.

FIG. 2 is a flow chart that is useful for understanding the operation of a home care monitoring system that is useful for understanding the present invention.

### DETAILED DESCRIPTION OF THE INVENTION

An embodiment in accordance with the present invention relates to a low cost system for monitoring people, such as those persons who are elderly or ill, in the safety and comfort of their own homes. In particular, a system can be used to remotely monitor persons in their homes over a secure communication link. Thus, the system can include a variety of equipment within a home of a person being monitored (client). For example, the system can include monitoring, processing and communication equipment. Notably, the system can be compliant with The Health Insurance Portability and Accountability Act of 1996 (HIPAA).

To establish monitoring parameters, the client's behavior can be analyzed and rules correlating to the client's behavior can be created. For example, if a particular client normally awakens at 7:00 A.M. and immediately proceeds straight to the kitchen to brew a pot of coffee, a rule can be established which represents a condition that the person usually enters the kitchen by 7:30 A.M. The behavior of the client then can be monitored to generate real time data correlating to the client's activities. The real time data can be processed and evaluated against the established rules, for instance using artificial intelligence.

For example, a motion detector can be provided within the client's kitchen to detect activity within the kitchen. If the motion detector has not detected activity in the kitchen by 7:30 A.M., an exception can be generated and sent to a monitoring station where a responsive action can be initiated. For instance, a telephone call can be placed to the home of the client to check on the client. If the client does not answer the telephone, a caregiver can be dispatched to the home. Importantly, other rules can be established for monitoring other parameters relating to the client or the client's home, such as environment parameters and medical parameters. Exceptions can be generated based on these other parameters as well.

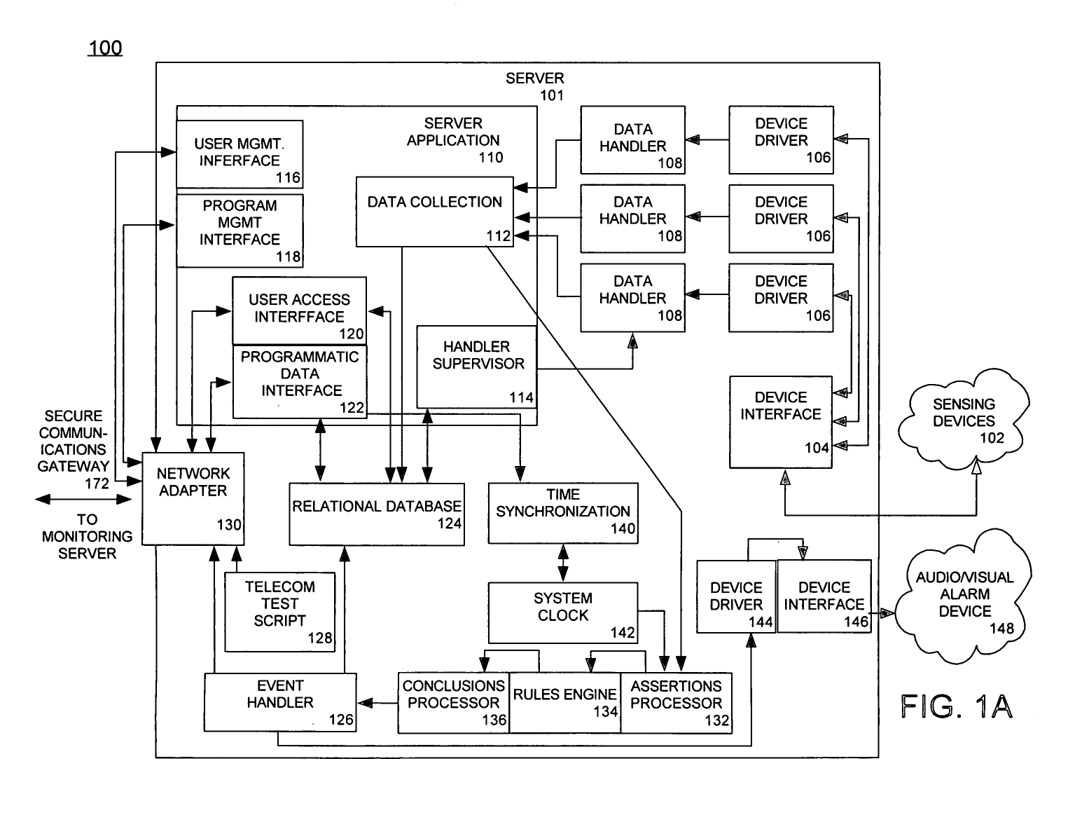

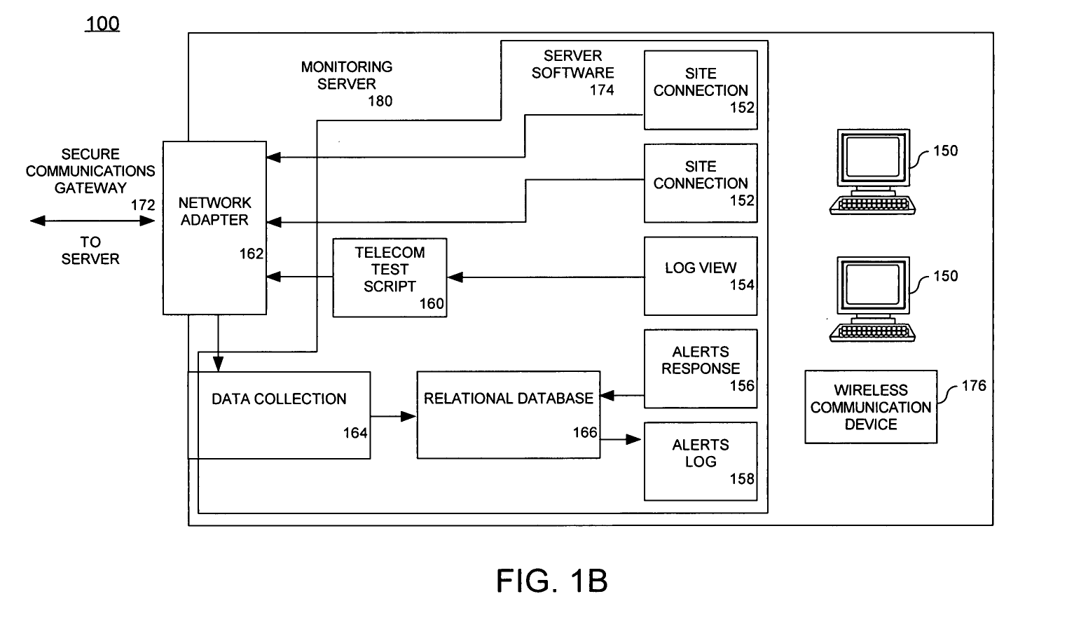

FIGS. 1A and 1B, taken together, represent a block diagram of an exemplary home care monitoring system (system) 100 that is useful for understanding the present invention. Referring to FIG. 1A, the system 100 can include an in-home server (server) 101, which can be a device having data communication and data processing capability. For example, the server can be a computer, an Internet appliance, or any other device which can receive and process sensor data and communicate with a remote monitoring station.

One or more sensing devices 102 can be provided to monitor at least one behavioral parameter associated with the client. The sensing devices 102 can generate data correlating to behavioral parameters being monitored and forward the data to the server 101. For example, the system 100 can include a microphone which detects acoustic signals, such as those signals associated with the client opening or closing a window, opening or closing a door, taking a shower, generating vocalizations, or any other acoustic signal associated with the client's behavior. Motion detectors and video cameras also can be provided within monitored portions of the client's home. The motion detectors can detect the motion of a person, such as the client, in a specific area. For example, motion detectors can be installed in the kitchen, a bathroom, a bedroom, or any other room or closet in the home.

The video cameras can monitor the client's behavior and environment. Moreover, the video cameras can be installed at any locations within, or about, the home as well. A client may want to limit the number of video cameras within the home, however, to maintain a certain level of privacy. Data generated by the video cameras can be stored, for instance to a datastore, as is discussed further below.

Other types of sensors also can be provided. For example, heat detectors can be provided to detect the presence of a person based on the temperature of the person's body. Sensors also can be provided to detect activation or deactivation of a lamp or other appliance. Still, a myriad of other devices are commercially available which can be used to monitor a client's behavioral patterns and such devices are understood to be within the scope of the present invention.

In addition to monitoring a client's behavior, detectors also can be provided to monitor environment parameters, such as conditions within or about the client's home. For example, there have been many instances of elderly persons parking their cars in their garages and forgetting to turn off the ignitions of the cars. This can cause a carbon monoxide buildup that seeps from the garage into the home and, tragically, carbon monoxide poisoning that causes death or serious injury to the occupants of the home. To reduce the risk of such an occurrence, a carbon monoxide detector can be included with system 100 to monitor carbon monoxide levels within the home and/or within the garage. Other types of environment detectors that can operate with the system 100 can include, for example, a smoke detector, a thermometer, a thermal couple, a water intrusion detector, a humidity detector, a moisture detector, a power failure detector, a power fluctuation detector, an earthquake detector, a barometer, and any other detector which can measure an environment condition.

The system 100 also can include at least one sensor or detector which monitors a medical parameter. For example, the system 100 can include a sensor which measures the client's blood pressure, pulse, blood glucose level, blood oxygen level, weight, heart rhythm, brain wave patterns, breathing patterns, and/or any other physical parameter associated with the client which can be measured. In one arrangement, the behavioral, environment and/or medical sensors can be integrated into a robot which roves within the client's home. The system 100 can also be used for medication reminders, for example using rules to generate pre-recorded or artificially generated voice messages through audio transducers and/or a video display attached to the system.

The data generated by the sensing devices 102 can be forwarded to a server 101. The server 101 can be a data processing device, such as a computer, a Web appliance, or any other device which can process data obtained from sensing devices 102. The server can operate a server application 110 to enable server functionality. There are many types of server software 110 commercially available, for example Apache available from the Apache Software Foundation, each of which is within the scope of the present invention.

One or more device interfaces 104, for example computer ports, can be provided. A device interface 104 can be, for example, a universal serial bus (USB), a serial port, a parallel port, an IEEE-1394 (FireWire) port, or any other port which can be used for communication between the sensing devices 102 and the server 101. Additionally, an RF receiver and preamplifier can be used as a device interface for wireless devices, for example those using X10, 802.11, blue tooth, or any other wireless technology. Notably, the device interfaces 104 can be internal or external to the server 101.

Device drivers 106 can be provided for controlling the sensing devices, as would be known by those skilled in the art of peripheral control. For example, device drivers can be provided for device controllers, video cameras, or any other sensing devices 102 being used with the system 100. Data handlers 108 can be provided to process data received from the sensing devices 102 for formatting and storage. For example, the data handlers can format the data using Extensible Markup Language (XML). Further, the data handlers can perform logical operations on the data using known computer languages, for example programming languages such as PHP: Hypertext preprocessor (PHP), Perl, Active Server Page (ASP), Java Server Page (JSP), or any other computer language that can perform logical operations on data.

Within the server application 110 a data collection module 112 can receive data from the data handlers 108. Again, the data collection module can operate using any language which can perform logical operations on the data. The data collection module can forward the collected data to a relational database 124 for storage. The relational database can be any database suitable for data storage. In one arrangement, the collected data can be stored with identifying parameters. For example, the data can be stored with an indicator that identifies which device generated the data, a time stamp, a location identifier, or any other information that may be relevant to the data. For example, the identity of persons inquiring into the data and the time the data was accessed also can be stored with the data.

The collected data also can be forwarded to an assertions processor 132. The assertions processor 132 can translate the data into executable assertions which can be evaluated by a rules engine 134. The rules engine. 134 can be any application which can evaluate assertions against established rules, for example an inference engine such as C Language Integrated Production System (CLIPS). For instance, if a rule states that a particular motion detector, such as a motion detector installed in a kitchen, should detect motion by 7:30 A.M. each morning, and such detection has not yet occurred at 7:30 on a particular morning, an assertion to that effect can be generated by the assertions processor 132. The rules engine 134 can evaluate the assertion and forward the evaluation to a conclusions processor 136. In another example, the rules engine 134 can analyze data from a video camera to detect unexpected behaviors in the home, such as a quick movement indicative of a fall or other undesirable circumstance. Notably, a number of rules/inference engines are commercially available and will be understood to be within the scope of the present invention.

Once a rule is triggered (i.e. the conditions or absence of conditions for a rule are met), the rules engine 134 can signal the triggering of the rule to a conclusions processor 136. Based on the information the conclusions processor 136 receives from the rules engine 134, the conclusions processor 136 can analyze the type or class of rule that has been triggered, and trigger an exception to be sent to an event handler 126. The event handler 126 can be a program that receives output from the conclusions processor 136 and dispatches or executes required actions. For example, the event handler 126 can forward the exception to the relational database 124 for storage. The event handler 126 also can forward the exception through a network adapter 130 to a remote location, such as the monitoring station, as will be discussed further below. The exception also can be forwarded to other modules within the server 101, for example, triggering local visual or audible alerts, voice messages or other actions. In one arrangement, event handler 126 can trigger an audible alert by forwarding an exception to a device driver 144. The device driver 144 can propagate audio/visual signals to an audio/visual alarm device 148 via a device interface 146.

A programmatic data interface 122, a user access interface 120, a program management interface 118 and a user management interface 116 can be provided within the server 101 to facilitate communications with the monitoring station. The programmatic data interface 122 can facilitate the machine-to-machine passing of data between the server 101 and the monitoring station. The user access interface 120 can provide user access, for example through a browser, of the data collected in the server 101 or a remotely located server. The program management interface 118, functioning at a machine to machine level, can interface management devices and applications from the monitoring station and the server 101. The user management interface 116 can provide an interface for users to manage devices and applications on the server 101 from the monitoring station. For example, users at the monitoring station can perform maintenance updates to software operation on the server 101, verify sensing devices 102 are properly functioning, activate or deactivate devices, activate, deactivate or modify rules, or perform any other type of user management function. The programmatic data interface 122, user access interface 120, program management interface 118 and user management interface 116 can be implemented as individual applications or as components of a larger, more complex, application.

Referring to FIGS 1A and 1B, the server 101 can be connected to a network, such as the Internet, a local area network (LAN), a wide are network (WAN), a public switched telephone network (PSTN), a public switched data network (PSDN), or any other type of communication network. The network connection can be established using the network adapter 130. The network adapter 130 can be a broadband modem, a conventional dial-up modem, an Ethernet adapter, or any other type of network adapter.

Telecom test scripts 128 can be automatically initiated at a periodic interval, for example every sixty seconds, to verify network communication is functioning properly. Further, a time synchronization program 140 can be executed each time the telecom test script 128 is successfully executed, collecting the atomic time from a public service server connected to the network to ensure the complete accuracy of time-dependent rules and system functions by setting a system clock 142.

Once network communication is established, the server 101 can securely open a secure communication link to a monitoring server 180 remotely located at the monitoring station. As with the server 101, the monitoring server 180 also can include a network adapter 162 which provides a communication link to the network. Again, the network adapter 162 can be a broadband modem, a conventional dial-up modem, an Ethernet adapter, or any other type of network adapter.

In addition to providing a communications path between the server 101 and monitoring server 180, the network connection can be used to establish a line of communication between the client and people or systems in other locations which are connected to the network. For example, communications links can be established between the client and the monitoring station or other persons using computers connected to the network. The network also can be used to provide an audio or video link, one example being voice over IP (VOIP), between the client and the monitoring station or other persons.

To insure privacy and reduce the risk of unauthorized access to the server 101 and monitoring server 180, the server 101 can communicate with the monitoring server 180 through a secure communications gateway 172, for example a secure sockets tunnel (SSL tunnel). Once the secure communications gateway is established, the monitoring server 180 can securely open a second communication link, for example a second SSL tunnel, within the secure communications gateway 172. This makes any function of the servers 101 unreachable from any point of the network (Internet) except from a particular, programmatically created, high-number port on the monitoring server 180. Notably, all communications between the servers can be encrypted, for example by using secure sockets layer (SSL) algorithms. Additionally, firewalls can be provided with both servers 101, 180 to provide additional security measures.

A telecom test script 160 can be initiated from the monitoring server 180 to verify proper communication between the monitoring station 180 and one or more servers. In one arrangement, the telecom test script 160 can be initiated at a periodic interval. A log view 154 can be used to view a log of test results generated by the telecom test script 160 as well as those generated by the telecom test script 128.

The monitoring server 180 can use the second communication link to maintain and manage the system 100. For example, the monitoring server 180 can evaluate generated data and data processing functions to insure proper operation of the system 100, consult or run reports on data and/or images stored on the server 101 or monitoring server 180, or perform any other maintenance or management functions. The remotely located monitoring server 180 can receive data and alerts from a plurality of servers, such as server 101, thereby enabling the monitoring server 180 to monitor any number of clients.

Data received through the network adapter 162 can be forwarded to a data collection module 164. The data collection module 164 can forward the data to a relational database 166 for storage. Exceptions contained in the received data then can be forwarded to an alerts log 158 and processed according to predetermined protocols. For example, the exceptions can be forwarded to a central relational database 166 for storage. The exceptions also can be forwarded to a user interface 150, for example, a computer workstation. Exceptions also can be forwarded to other devices, for example a wireless communications device 176. The wireless communications device 176 can be, for example, a mobile telephone, a pager, a personal digital assistant (PDA), or any other wireless communication device. A user monitoring the exceptions can respond to an exception, for example using the user interface 150, and the response can be forwarded to the relational database 166 via an alert response module 156.

A user also can utilize a user interface 150 and site connection modules 152 to obtain access to server 101 via the secure communications gateway 172, for example using the second communication link. In this manner the user can securely manage a plurality of client systems from a remote location. For example, a user can perform server maintenance or initiate real time monitoring of data being received from the sensing devices 102. Further, the user can view data stored on the server 101. For example, a user can receive and evaluate data from the sensors 102, such as in a situation where it is suspected that the client has been injured in some way or is otherwise incapacitated. For example, the user can view video data to determine if a client has fallen or is unconscious.

Further, the user can attempt to contact the client using the VOIP communication link. If the client is incapacitated, the user can initiate an emergency call to expedite emergency intervention by initiating an outbound call from the client's home. For example, a client-phone localized emergency call (“911”) can be placed to facilitate location identification of the client's home by emergency care givers. The outbound call can be placed using a computer modem and patching the modem to the server 101 over the VOIP link. The VOIP link also can allow verbal communication between the user and the appropriate emergency call operator to describe the situation as if he or she were calling from the client phone.

The user also can issue remote commands from the monitoring station to the server 101. For example, the user can control from the monitoring station an appliance, a lamp, a sensor or a medical device which is operatively connected to the server 101. This can be a particularly useful feature for client's having reduced levels of memory retention. For instance, appliances can be fitted with controls that can be remotely operated from the monitoring station. Thus, in the case that a client forgets to turn off a stove after cooking, the user can send a command which causes the stove to be turned off. In another arrangement, the server 101 can be programmed to turn off the stove after a predetermined duration of operation. Still, there are many other appliances that can be controlled from a remote location, and the present invention is not limited to the examples presented herein.

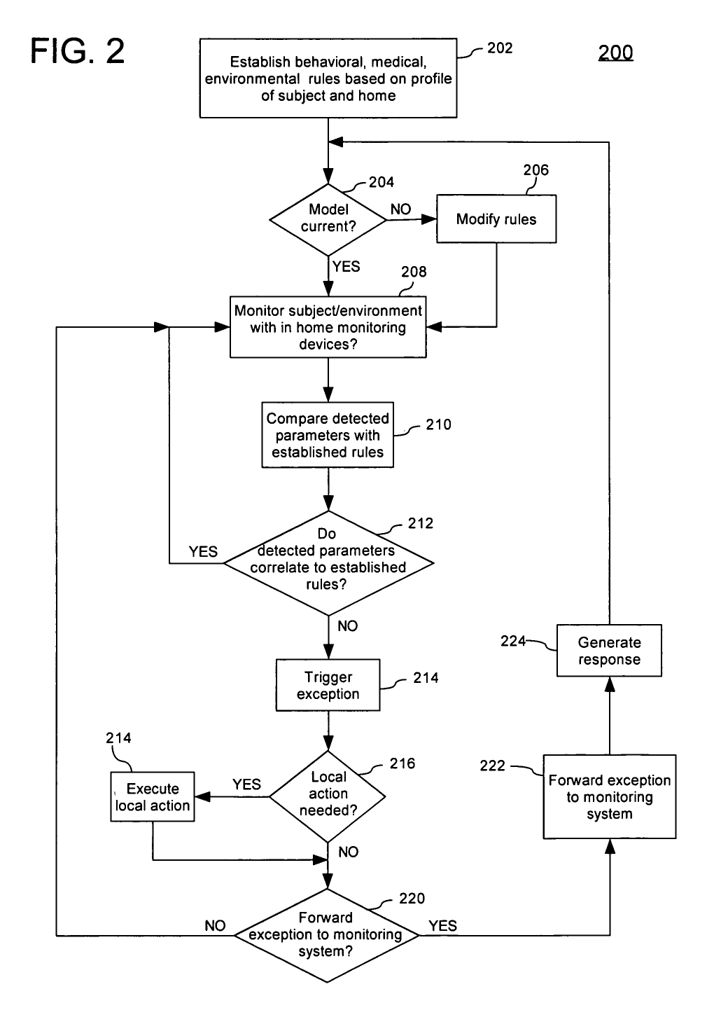

Referring to FIG. 2, a flowchart 200 is shown which presents an exemplary method for use and operation of a homecare monitoring system. Referring to step 202, behavioral rules based on the behavioral profiles of a subject (client) can be established. The behavioral rules can represent any aspect of the client's behavior, for example, the time the client wakes up each morning, the time the client goes to bed each night, how frequently the client visits the restroom, at which time the client enters a kitchen, whether and at what time the client is expected to exit his residency, or any other behavior for which a rule can be applied. In alternate arrangements, rules for environment parameters and a client's medical parameters can be established.

For example, rules can be established for acceptable carbon monoxide levels in the air, acceptable smoke levels, acceptable temperature, whether water has intruded into the home, an acceptable moisture level, acceptable times for opening and closing of windows and doors, or any other parameter affecting the client's environment. Examples of rules that can be established for evaluating various medical parameters can include acceptable ranges of blood pressure, pulse rate, blood glucose levels, blood oxygen levels, physical weight, heart rhythms, brain waves, respiration patterns, or any other medical parameter that can be monitored. Rules also can be established to verify whether regularly expected signals are received from sensing devices, for instance, whether a blood pressure, pulse and respiration readings have taken place at their scheduled times. Additionally, rules representing the time and conditions under which the client should take prescribed medication also can be established.

Referring to decision box 204, if the rules established for the client are not current, the rules can be modified, as shown in step 206. For example, software implementing the rules in the server 101 or monitoring server 180 can be modified or updated.

Referring to step 208 the client can be monitored in the home using monitoring devices. Data from the devices can be forwarded to a server where the client's behavior or medical conditions, or the home environment can be compared to established rules, as shown in step 210. For example, whether the client follows a known behavioral pattern such as entering the kitchen by a certain time can be evaluated. The medical parameters associated with the client can be evaluated as well, for example pulse, blood pressure, etc.

Importantly, client monitoring rules can be modified or suspended at certain times, for example when the client's health status changes, when the client is away from home or when new persons or animals are added to the household. The changes to the rules can be implemented in any number of ways. For instance, the client, or the client's caregiver, can telephone a monitoring center, or log into the server, and provide updated client information. When providing updated information, the client or caregiver can provide a specific code or password for user validation. Voice identification also can be performed when the client or caregiver verbally update the client information. In the case that the verbal updates are provided over a telephone, the telephone number from which the call originates can be authenticated using caller identification. Still, there are numerous other security protocols that are known to the skilled artisan, and such protocols are within the scope of the present invention.

Referring to decision box 210, detected parameters can be compared with established rules. If the detected parameters correlate to the established rules, monitoring can continue. If the detected parameters do not correlate to the established rules, an exception can be triggered, as shown in decision box 212 and step 214. Referring to decision box 216, a determination can be made whether only a local action is needed to respond to the exception. If only a local action is needed, a local action can be executed by the server, as shown in step 218. For example, a pre-recorded reminder message can be played, such as an audio recording played through loudspeakers and/or an audio/visual recording can be presented on a television. Such a pre-recorded message can be especially beneficial if a client has missed a regularly scheduled medical test, for example a blood pressure reading, pulse measurement or respiration reading. Pre-recorded messages also can be played to remind a client to take an action in response to results from the medical test or to remind the client to take medication at a pre-determined time. Visible and audible alarms also can be generated, for instance if there is a medical condition, behavioral parameter or environment parameter detected that is particularly concerning.

Proceeding to decision box 220, a determination can be made whether an exception should be forwarded to a monitoring system. If so, the exception can be sent, as shown in step 222, and the monitoring system can generate a response as shown in step 224. For example, a response can be a telephone call to the home to verify whether the client needs assistance. If assistance is needed or there is no response from the client, a caregiver can be dispatched to the home. In some instances the client may be in need of professional medical attention. In such cases an ambulance can be dispatched or any other necessary medical attention can be provided. Referring again to decision box 204, the rules established for the client can be re-evaluated and modified or updated as required. New rules also can be established if needed.

The present invention can be realized in hardware, software, or a combination of hardware and software. The present invention can be realized in a centralized fashion in one computer system, or in a distributed fashion where different elements are spread across several interconnected computer systems. Any kind of computer system or other apparatus adapted for carrying out the methods described herein is suited. A typical combination of hardware and software can be a general purpose computer system with a computer program that, when being loaded and executed, controls the computer system such that it carries out the methods described herein.

The present invention also can be embedded in a computer program product, which comprises all the features enabling the implementation of the methods described herein, and which when loaded in a computer system is able to carry out these methods. Computer program in the present context means any expression, in any language, code or notation, of a set of instructions intended to cause a system having an information processing capability to perform a particular function either directly or after either or both of the following: a) conversion to another language, code or notation; b) reproduction in a different material form.

This invention can be embodied in other forms without departing from the spirit or essential attributes thereof. Accordingly, reference should be made to the following claims, rather than to the foregoing specification, as indicating the scope of the invention.

What is claimed is:

1. A method for in-home monitoring comprising the steps of:
monitoring at least one behavioral parameter associated with a person;
comparing the behavioral parameter to at least one pre-determined rule which is based upon a behavioral profile;
triggering an exception if the behavioral parameter does not match the behavioral profile; and
initiating at least one action responsive to the exception.

2. The method according to claim 1, said initiating at least one action step further comprising the step of forwarding the exception to a monitoring system;

3. The method according to claim 1, further comprising the step of empirically determining the behavioral profile based upon behavioral patterns of the person.

4. The method according to claim 1, wherein said comparing step further includes the step of analyzing the behavioral parameter using artificial intelligence.

5. The method according to claim 4, wherein the artificial intelligence is implemented with an inference engine.

6. The method according to claim 1, wherein the behavioral parameter is selected from the group consisting of an acoustic signal, a movement of a person, a location of a person, an opening of a window, a closing of a window, an opening of a door, a closing of a door, an activation of an appliance, a deactivation of an appliance, an activation of a light, and a deactivation of al light.

7. The method according to claim 1, wherein data representing the behavioral parameter is wirelessly propagated from a sensing device to a device interface.

8. The method according to claim 1, further comprising the steps of:
monitoring at least one environment parameter;
comparing the environment parameter to at least one pre-determined environment rule; and
triggering the exception if the environment parameter correlates to an environment condition that has been pre-defined to trigger the exception.

9. The method according to claim 8, wherein the environment parameter is selected from the group consisting of a carbon monoxide level, a smoke level, a temperature, an amount of water intrusion, a moisture level, a power failure, a weather condition, an earthquake, an acoustic signal, an opening of a window, a closing of a window, an opening of a door, a closing of a door, and a detected motion.

10. The method according to claim 1, further comprising the steps of:
monitoring at least one medical parameter;
comparing the medical parameter to at least one pre-determined medical rule; and
triggering the exception if the medical parameter correlates to a medical condition pre-defined to trigger the exception.

11. The method according to claim 10, wherein the medical parameter is selected from the group consisting of a blood pressure, a pulse, a blood glucose level, a blood oxygen level, a weight, a heart rhythm, a brain wave, and a breathing pattern.

12. The method according to claim 1, further comprising the step of providing a processing device within a home of the person wherein the processing device provides the monitored behavioral parameters to at least one monitoring station located outside of the home.

13. The method according to claim 1, further comprising the step of generating at least one medication reminder.

14. The method according to claim 1, wherein said step of initiating at least one action comprises generating a client-phone localized emergency call.

15. A system for in-home monitoring comprising:
at least one sensor for monitoring at least one behavioral parameter associated with a person and generating correlating data;
at least one processing device; and
at least one software application executing on said processing device, said software application comparing said data to at least one pre-determined rule which is based upon a behavioral profile and triggering an exception if said data correlates to a condition pre-defined to trigger said exception.

16. The system of claim 15, further comprising a device interface for receiving said data and forwarding said data to said processing device.

17. The system of claim 16, wherein said sensor wirelessly propagates said data to said device interface.

18. The system of claim 15, further comprising a communication link for communicating with a monitoring station.

19. The system of claim 18, wherein said processing device forwards said exception to said monitoring station via said communication link.

20. The system of claim 18, wherein said system receives remote commands from said monitoring station.

21. The system of claim 20, wherein said remote commands control at least one item selected from the group consisting of an appliance, a lamp, a sensor and a medical device.

22. The system of claim 18, wherein said monitoring station initializes a client-phone localized emergency call by sending a command over said communication link.

23. The system of claim 15, wherein said sensor is selected from the group consisting of a microphone, a video camera, an infrared motion detector, a carbon monoxide detector, a smoke detector, a fire detector, a water intrusion detector, a power failure detector, a door contact and a window contact.

24. The system of claim 15, wherein said sensor monitors a physical attribute of a person.

25. The system of claim 24, wherein said physical attribute is selected from the group consisting of a blood pressure, a pulse, a blood glucose level, a blood oxygen level, a weight, a heart rhythm, a brain wave, and a breathing pattern.

26. The system of claim 15, further comprising at least one roving robot which monitors at least one of the behavioral parameters, environment parameters and a physical attribute of a person.
